(19) United States
(12) Patent Application Publication
(10) Pub. No.: US 2025/0017548 A1
(43) Pub. Date: Jan. 16, 2025

(54) METHOD AND SYSTEM FOR AUTOMATIC SCAN SUBJECT POSITIONING

(71) Applicant: GE Precision Healthcare LLC, Waukesha, WI (US)

(72) Inventors: Lu Shen, Beijing (CN); Li Fang, Beijing (CN); Dong Wei, Beijing (CN)

(21) Appl. No.: 18/771,774

(22) Filed: Jul. 12, 2024

(30) Foreign Application Priority Data
Jul. 13, 2023 (CN) 202310865670.9

(51) Int. Cl. A61B 6/00; A61B 6/03; A61B 6/04; A61B 6/40; A61B 6/46; G16H 10/60; G16H 30/20

(52) U.S. Cl. CPC A61B 6/545; A61B 6/03; A61B 6/0407; A61B 6/0487; A61B 6/4078; A61B 6/4085; A61B 6/46; G16H 10/60; G16H 30/20

(57) ABSTRACT

Disclosed are a method and system for automatic scan subject positioning in medical imaging. The method includes the following steps: acquiring scan information of a scan subject to be imaged and determining a scan subject scanning feature; identifying, on the basis of the scan subject scanning feature, a corresponding offset characteristic value from a mapping table between scanning features and offset characteristic values; and adjusting, on the basis of the corresponding offset characteristic value, a target position of the scan subject during automatic scan subject positioning.

### CROSS-REFERENCE TO RELATED APPLICATIONS

This application claims priority to Chinese Application No. 202310865670.9, filed on Jul. 13, 2023, the disclosure of which is incorporated herein by reference in its entirety.

### TECHNICAL FIELD

The present invention relates to the technical field of medical imaging systems, and relates in particular to a method and system for automatic scan subject positioning. The present invention also particularly relates to a processor that performs the above method and a computer-readable storage medium that stores a computer program capable of implementing the above method.

### BACKGROUND

Non-invasive radiographic techniques allow images of the internal anatomy of a scan subject (typically including a patient) to be acquired without performing any invasive operation on the scan subject. In particular, techniques such as computed tomography (CT) use various physical principles (such as differential transmission of X-rays passing through a target volume) to acquire image data and construct tomographic images (e.g., three-dimensional representations of the interior of a human body or another imaged structure). In modern CT imaging systems, a gantry includes an annular frame provided with an X-ray tube on one side and a detector on the opposite side. The frame rotates around a patient positioned on a workbench, thereby producing thousands of cross-sectional views of the patient in one scan. In order to efficiently use these imaging techniques and achieve better image quality, a scan subject must be properly positioned inside the imaging system, typically including aligning the physical center of a scanned site of the scan subject with the isocenter of a medical imaging device, i.e., “centering”.

In current medical imaging systems, a 3D depth camera is used to assist automatic positioning of a scan subject. Due to the limited accuracy of the body contour acquired by the 3D depth camera, the accuracy of centering the scan subject at the isocenter of the medical imaging device needs to be improved. Therefore, there is a need in the art for optimizing current automatic positioning.

### SUMMARY

An objective of the present invention is to overcome the above and/or other problems in the prior art, and the present invention can optimize automatic positioning in a medical imaging system, thereby improving centering accuracy.

According to a first aspect of the present invention, a method for automatic scan subject positioning in medical imaging is provided. The method comprises the following steps: acquiring scan information of a scan subject to be imaged and determining a scan subject scanning feature; identifying, on the basis of the scan subject scanning feature, a corresponding offset characteristic value from a mapping table between scanning features and offset characteristic values; and adjusting, on the basis of the corresponding offset characteristic value, a target position of the scan subject in the automatic scan subject positioning.

According to a second aspect of the present invention, a medical imaging system is provided. The system comprises: a medical imaging apparatus, configured to perform scanning and imaging on a scan subject; a scan subject moving table, configured to support the scan subject and movable in a horizontal direction and a vertical direction, the horizontal direction comprising a transverse direction and a longitudinal direction, wherein the scan subject moving table is moved in the longitudinal direction during the scanning and imaging; a 3D depth camera, configured to acquire a body contour of the scan subject; an automatic positioning module, configured to perform automatic positioning of the scan subject according to scan information of the scan subject and the acquired body contour; and a positioning optimization module, configured to perform the following: acquiring the scan information and determining a scan subject scanning feature; identifying, on the basis of the scan subject scanning feature, a corresponding offset characteristic value from a mapping table between scanning features and offset characteristic values; and adjusting, on the basis of the corresponding offset characteristic value, a target position of the scan subject in the automatic positioning.

Preferably, the mapping table is established via the following steps: acquiring historical imaging scan data, the historical imaging scan data comprising historical scan information and historical offsets associated with a plurality of scanning procedures; determining, from historical scan information of each scanning procedure, a scanning feature of a scan subject of the scanning procedure, the scanning feature of the scan subject comprising an anatomical structure category of the scan subject; for the same scanning feature, calculating a statistical value of historical offsets thereof, and determining the statistical value or an inverse of the statistical value as an offset characteristic value corresponding to the scanning feature; and generating a mapping table between different scanning features and offset characteristic values thereof.

Preferably, the historical scan information comprises a scanning protocol and/or a scanning descriptor, and the anatomical structure category of the scan subject is determined on the basis of the scanning protocol and/or the scanning descriptor.

Preferably, the scanning feature further comprises an age range, sex, and/or a body type of the scan subject, and the historical scan information comprises information for determining the age range, the sex, and/or the body type of the scan subject.

Preferably, the historical scan information further comprises a positioning identifier for identifying whether manual positioning or automatic positioning is used in each scanning procedure, wherein the mapping table is generated only for scanning procedures using automatic positioning among the plurality of scanning procedures.

Preferably, the scanning feature comprises an anatomical structure category, the scan information comprises a scanning protocol and/or a scanning descriptor, and the step of determining a scan subject scanning feature comprises: determining an anatomical structure category of the scan subject on the basis of the scanning protocol and/or the scanning descriptor.

Preferably, the scanning feature comprises an anatomical structure category, the scan information comprises a scan range, and the step of determining a scan subject scanning feature comprises: identifying a plurality of landmarks of the scan subject by using a 3D camera, and determining an anatomical structure category of the scan subject on the basis of a positional relationship between at least one of the plurality of landmarks and the scan range.

Preferably, the scanning feature further comprises an age range, sex, and/or a body type of the scan subject, and the scan information comprises information for determining the age range, the sex, and/or the body type of the scan subject.

Preferably, the information for determining the body type of the scan subject comprises BMI of the scan subject, or a body contour of the scan subject acquired by using a 3D camera.

Preferably, the age ranges of the scan subject comprise adult and pediatric, and the body types of the scan subject comprise obese, normal, and thin.

Preferably, the anatomical structure categories comprise head, chest, abdomen-pelvis, pelvis, lumbar spine, and heart.

Preferably, the method further comprises the following steps: performing scanning and imaging on an anatomical structure of the scan subject according to the scan information by using the medical imaging system, so as to generate an image file, wherein the scan subject is moved in a horizontal direction during the scanning and imaging; processing the image file to acquire a center point of the anatomical structure; calculating a current offset in a vertical direction between the center point of the anatomical structure and an isocenter point of the medical imaging system; and by using the scan subject scanning feature and the current offset, adjusting the offset characteristic value corresponding to the scan subject scanning feature in the mapping table.

Preferably, the adjusting step comprises: performing statistical computation on the offset characteristic value corresponding to the scan subject scanning feature and the current offset, and determining a new offset characteristic value.

Preferably, the scan subject to be imaged is movable in a horizontal direction and a vertical direction via a scan subject moving table, and the horizontal direction comprises a transverse direction and a longitudinal direction, wherein the step of adjusting a target position of the scan subject comprises adjusting a target position of the scan subject in the vertical direction or the transverse direction.

According to a third aspect of the present invention, a processor is provided, and is configured to perform the method described above.

According to a fourth aspect of the present invention, a computer-readable storage medium having a computer program stored thereon is provided, wherein the program, when executed by a processor, implements the steps of the method described above.

### BRIEF DESCRIPTION OF THE DRAWINGS

The present invention can be better understood by means of the description of the exemplary embodiments of the present invention in conjunction with the drawings, in which:

FIG. 1 shows an exemplary CT imaging system.

FIG. 2 shows an exemplary imaging system similar to the CT imaging system in FIG. 1.

FIG. 3 is a flowchart of a method for optimizing automatic patient positioning according to an exemplary embodiment of the present invention.

FIG. 4 shows a schematic diagram of a mapping table between scanning features and offset characteristic values.

FIG. 5 shows an example of a mapping table.

FIG. 6 shows a flowchart of establishment of a mapping table according to an embodiment of the present invention.

FIG. 7 shows a screenshot of an exemplary table of historical imaging scan data acquired during establishment of a mapping table.

FIG. 8 shows an example of a mapping table between scanning features and offset characteristic values.

FIG. 9 shows a schematic diagram of a scan field of view (SFOV) of an X-ray fan beam or cone beam.

FIG. 10 shows an image example of a 90° scout scan.

FIG. 11 shows an example of human body landmarks and a schematic diagram of a positional relationship between landmarks and a scan range.

FIG. 12 to FIG. 16 respectively show a plurality of cases of a positional relationship between at least one landmark and a scan range.

FIG. 17 shows a flowchart of adaptive optimization of a mapping table according to an embodiment of the present invention.

FIG. 18 exemplarily shows a schematic diagram of a mapping table generated according to the method described above and adaptive optimization.

FIG. 19 shows an example of an electronic device according to an embodiment of the present invention.

In the accompanying drawings, similar components and/or features may have the same numerical reference signs. Further, components of the same type may be distinguished by letters following the reference sign, and the letters may be used for distinguishing between similar components and/or features. If only a first numerical reference sign is used in the specification, the description is applicable to any similar component and/or feature having the same first numerical reference sign irrespective of the subscript of the letter.

### DETAILED DESCRIPTION

Specific embodiments of the present invention will be described below. It should be noted that in the specific description of said embodiments, for the sake of brevity and conciseness, the present description cannot describe all of the features of the actual embodiments in detail. It should be understood that in the actual implementation process of any embodiment, just as in the process of any one engineering project or design project, a variety of specific decisions are often made to achieve specific goals of the developer and to meet system-related or business-related constraints, which may also vary from one embodiment to another. Furthermore, it should also be understood that although efforts made in such development processes may be complex and tedious, for a person of ordinary skill in the art related to the content disclosed in the present invention, some design, manufacture, or production changes made on the basis of the technical content disclosed in the present disclosure are only common technical means, and should not be construed as the content of the present disclosure being insufficient.

Unless defined otherwise, technical terms or scientific terms used in the claims and description should have the usual meanings that are understood by those of ordinary skill in the technical field to which the present invention belongs. The terms “first,” “second,” and the like used in the description and claims of the patent application of the present invention do not denote any order, quantity or importance, but are merely intended to distinguish between different constituents. The terms “one,” “a/an,” and the like do not denote a limitation of quantity, but rather denote the presence of at least one of the referenced items. The terms “include,” “comprise,” and the like are intended to mean that an element or article that appears before “include” or “comprise” encompasses elements or articles and equivalent elements that are listed after “include” or “comprise,” and do not exclude other elements or articles. The terms “connect,” “connected,” and the like are not limited to physical or mechanical connection, and are not limited to direct or indirect connection. The phrase “scan subject” generally includes, but is not limited to, a patient, an animal, or other subjects examined by a medical imaging device.

The anatomy, position, and orientation of the patient all affect a radiographic result. An inappropriate position and/or orientation of the patient during or before a scan can significantly affect both the image noise and the radiation dose received by the patient. As an example, placing the patient in an off-center position may result in imaging artifacts and unnecessary radiation exposure to a more sensitive region on the body.

The desired patient position and orientation (e.g., posture) in radiological examination are based on the body part to be imaged, a suspected defect or disease, and patient conditions, and the positioning scheme is determined by a radiologist. Then, a technician operating the imaging system implements the specified scheme to acquire accurate diagnostic information and reduce X-ray exposure for the patient. In addition, the technician may manually adjust the height and the horizontal position of a scan workbench on which the patient is positioned, so as to align the patient for radiological examination. However, the technician may make a technical error due to, for example, the high workload and low efficiency of manual positioning. For example, the technical error may result in images acquired during the radiological examination having issues of over-exposure, under-exposure, or incorrect positioning of the patient. Thus, the radiologist may decide to reject and repeat the scan for an accurate diagnosis. In such an example, an immediate second radiograph may be required when the patient is available. Alternatively, the patient may have to return for an additional appointment so as to be scanned again. Both options increase the patient's discomfort, the patient's exposure to radiation, the cognitive stress on a scanning operator, and the amount of time until diagnosis.

Accordingly, automatic patient positioning techniques have been employed to speed up the workflow of radiological examination. The techniques include integrating time-of-flight (ToF) or a depth camera into a radiological examination room. Prior to radiographic imaging, the ToF or depth camera can be used to generate a 3D depth image of the patient. The 3D depth image can be used to determine the patient anatomy, including anatomical key points, a body contour, a body volume/thickness, and the position/orientation of the patient relative to the workbench. Then, a horizontal scan range of the patient anatomy can be determined on the basis of the patient anatomy and an anatomical structure to be imaged, and a scan height (i.e., the position in the vertical direction) for the patient anatomy can be approximately determined on the basis of whether the anatomical structure to be imaged is the head or the body. It is desirable to center the anatomical structure so that the anatomical structure is aligned with the CT isocenter.

Currently, there are two methods for centering and positioning a patient: (1) manually moving the workbench via buttons on an interactive control panel, so that the patient is visually centered; and (2) automatically positioning the patient on the basis of a body contour captured by a 3D depth camera, the automatic centering being based on an average body contour center of all scout scan ranges.

However, due to the limited accuracy of the body contour acquired by the 3D depth camera, centering (especially centering in the vertical direction) of certain anatomical structures (such as the chest, the lumbar spine, the ankle, the musculoskeletal (MSK) system, etc.) or special patients (pediatric or obese patients) is not good enough. For example, when it is desired to image the chest of an obese patient, automatic centering in automatic patient positioning is based on an average body contour center of all scout scan ranges, so that even after the automatic centering, the center of the chest of the obese patient deviates significantly from the CT isocenter point in the vertical direction. Thus, although automatic patient positioning is performed before a scan, it is still possible for the scan to be discarded and repeated.

Thus, according to embodiments disclosed herein, a method and system for automatic scan subject positioning in medical imaging are provided. In one embodiment, the method may include: acquiring scan information of a scan subject to be imaged and determining a scan subject scanning feature; identifying, on the basis of the scan subject scanning feature, a corresponding offset characteristic value from a mapping table between scanning features and offset characteristic values; and adjusting, on the basis of the corresponding offset characteristic value, a target position of the scan subject in the automatic scan subject positioning.

Although the operating environment of the present invention is described with respect to X-ray computed tomography (CT) systems, it should be understood that the technical solution of the present invention is also applicable to other medical imaging systems and/or medical imaging devices that utilize imaging bore holes and workbenches, such as X-ray imaging systems, magnetic resonance imaging (MRI) systems, positron emission tomography (PET) imaging systems, single-photon emission computed tomography (SPECT) imaging systems, and combinations thereof (e.g., multi-modal imaging systems such as PET/CT and PET/MR imaging system). The discussion on CT imaging modalities in the present invention is provided only as an example of one suitable imaging modality.

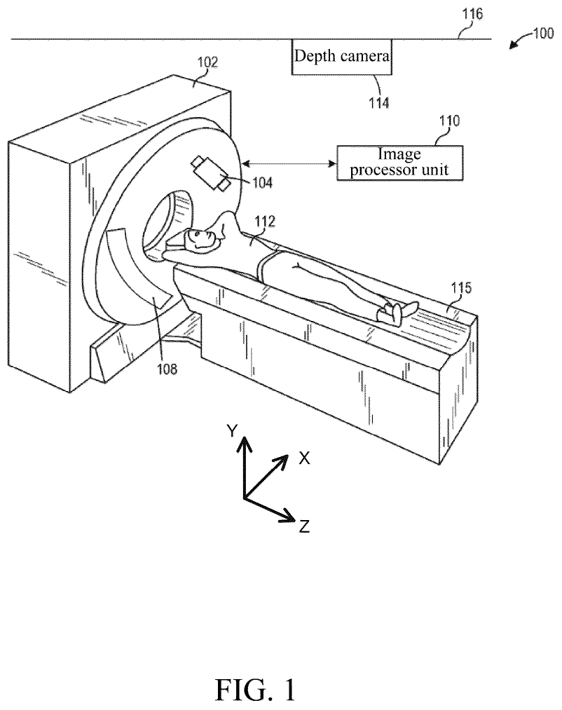

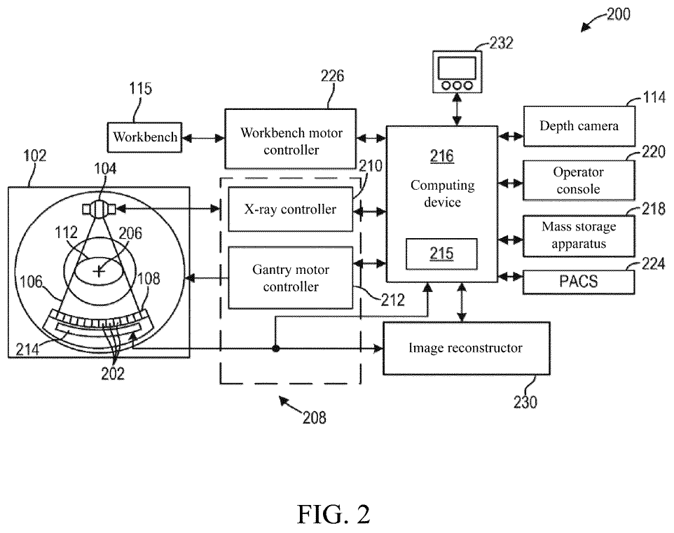

FIG. 1 shows an exemplary CT imaging system 100. Specifically, the CT imaging system 100 is configured to image a subject under examination 112 (such as a patient, an inanimate object, or one or more manufactured components or industrial components) and/or a foreign object (such as an implant, a stent, and/or a contrast agent present in the body). Throughout the present disclosure, the terms “subject under examination” and “patient” may be used interchangeably, and it should be understood that, at least in some examples, a patient is a type of subject under examination that can be imaged by a CT imaging system, and that a subject under examination may include the patient. In one embodiment, the CT imaging system 100 includes a gantry 102, which in turn may further include at least one X-ray radiation source 104. The at least one X-ray radiation source is configured to project an X-ray radiation beam (or X-ray) 106 (see FIG. 2) for imaging a patient. Specifically, the X-ray radiation source 104 is configured to project the X-ray 106 toward a detector array 108 positioned on the opposite side of the gantry 102. Although FIG. 1 illustrates only one X-ray radiation source 104, in certain embodiments, a plurality of radiation sources may be used to project a plurality of X-rays 106 toward a plurality of detectors, so as to acquire projection data corresponding to the patient at different energy levels.

In some embodiments, the X-ray radiation source 104 projects the X-ray fan beam or cone beam 106. The X-ray fan or cone beam is collimated to be located within an XY plane of a Cartesian coordinate system, and the plane is generally referred to as an “imaging plane” or a “scanning plane”. The X-ray beam 106 passes through the subject under examination 112. The X-ray beam 106, after being attenuated by the subject under examination 112, is incident on the detector array 108. The intensity of the attenuated radiation beam received at the detector array 108 depends on the attenuation of the X-ray 106 by the subject under examination 112. Each detector element of the detector array 108 produces a separate electrical signal that serves as a measure of the intensity of the beam at the detector position. Intensity measurements from all detectors are separately acquired to generate a transmission distribution.

In third-generation CT imaging systems, the gantry 102 is used to rotate the X-ray radiation source 104 and the detector array 108 within the imaging plane around the subject under examination 112, so that the angle at which the X-ray beam 106 intersects with the subject under examination 112 is constantly changing. A full gantry rotation occurs when the gantry 102 completes a full 360-degree rotation. A set of X-ray attenuation measurements (e.g., projection data) from the detector array 108 at one gantry angle is referred to as a “view”. Thus, the view represents each incremental position of the gantry 102. A “scan” of the subject under examination 112 includes a set of views made at different gantry angles or viewing angles during one rotation of the X-ray radiation source 104 and the detector array 108.

In an axial scan, projection data is processed to construct an image corresponding to a two-dimensional slice captured through the subject under examination 112. A method for reconstructing an image from a set of projection data is referred to as a filtered back projection technique in the art. The method converts an attenuation measurement from a scan into an integer referred to as “CT number” or “Hounsfield unit” (HU), the integer being used to control, for example, the brightness of a corresponding pixel on a cathode ray tube display.

In some examples, the CT imaging system 100 may include a depth camera 114 positioned on or outside the gantry 102. As shown, the depth camera 114 is mounted on a ceiling panel 116 positioned above the subject under examination 112, and is oriented to image the subject under examination 112 when the subject is at least partially outside the gantry 102. The depth camera 114 may include one or more light sensors, including one or more visible light sensors and/or one or more infrared (IR) light sensors. In some embodiments, the one or more IR sensors may include one or more sensors in a near-IR range and a far-IR range, thereby implementing thermal imaging. In some embodiments, the depth camera 114 may further include an IR light source. The light sensor may be any 3D depth sensor (such as a time-of-flight (ToF) sensor, a stereo sensor, or a structured light depth sensor) operable to generate a 3D depth image, while in other embodiments, the light sensor may be a two-dimensional (2D) sensor operable to generate a 2D image. In some such embodiments, a 2D light sensor may be used to infer a depth based on an understanding of light reflection phenomena, so as to estimate a 3D depth. Regardless of whether the light sensor is a 3D depth sensor or a 2D sensor, the depth camera 114 may be configured to output a signal for encoding an image to a suitable interface. The interface may be configured to receive, from the depth camera 114, the signal for encoding the image. In other examples, the depth camera 114 may further include other components, such as a microphone, so that the depth camera can receive and analyze directional and/or non-directional sound from the observed subject under examination and/or other sources.

In some embodiments, the CT imaging system 100 further includes an image processing unit 110, configured to reconstruct an image of a target volume of a patient by using a suitable reconstruction method (such as an iterative or analytical image reconstruction method). For example, the image processing unit 110 may reconstruct an image of a target volume of a patient by using an analytical image reconstruction method such as filtered back projection (FBP). As another example, the image processing unit 110 may reconstruct an image of a target volume of a patient by using an iterative image reconstruction method (such as adaptive statistical iterative reconstruction (ASIR), conjugate gradient (CG), maximum likelihood expectation maximization (MLEM), model-based iterative reconstruction (MBIR), or the like).

As used herein, the phrase “reconstructed image” is not intended to exclude embodiments of the present invention in which data representing an image is generated rather than a viewable image. Thus, as used herein, the term “image” broadly refers to both a viewable image and data representing a viewable image. However, many embodiments generate (or are configured to generate) at least one viewable image.

The CT imaging system 100 further includes a workbench 115, and the subject under examination 112 is positioned on the workbench to facilitate imaging. The workbench 115 may be electrically powered so as to be movable in a horizontal direction (XZ plane) and a vertical direction (Y). The horizontal direction includes a transverse direction (the X direction of the medical imaging system) and a longitudinal direction (the Z direction of the medical imaging system). During scanning and imaging, the workbench 111 is moved in the longitudinal direction. Accordingly, the workbench 115 may include a motor and a motor controller, as will be explained below with respect to FIG. 2. The workbench motor controller moves the workbench 115 by adjusting the motor, so as to properly position the subject under examination in the gantry 102 to acquire projection data corresponding to a target volume of the subject under examination. The workbench motor controller may adjust the height of the workbench 115 (e.g., a vertical position relative to the ground on which the workbench is located) and a horizontal position of the workbench 115 (e.g., a horizontal position in the transverse direction or the longitudinal direction).

FIG. 2 shows an exemplary imaging system 200 similar to the CT imaging system 100 in FIG. 1. In one embodiment, the imaging system 200 includes the detector array 108 (see FIG. 1). The detector array 108 further includes a plurality of detector elements 202, which together acquire the X-ray beam 106 (see FIG. 1) passing through the subject under examination 112 to acquire corresponding projection data. Therefore, in one embodiment, the detector array 108 is fabricated in a multi-slice configuration including a plurality of rows of units or detector elements 202. In such a configuration, one or more additional rows of detector elements 202 are arranged in a parallel configuration to acquire projection data. In some examples, an individual detector in the detector array 108 or the detector elements 202 may include a photon counting detector that registers interactions of individual photons into one or more energy bins. It should be understood that the methods described herein may also be implemented using an energy integration detector.

In certain embodiments, the imaging system 200 is configured to traverse different angular positions around the subject under examination 112 to acquire required projection data. Therefore, the gantry 102 and components mounted thereon can be configured to rotate about a center of rotation 206 to acquire, for example, projection data at different energy levels. Alternatively, in embodiments in which the projection angle with respect to the subject under examination 112 changes over time, the mounted components may be configured to move along a substantially curved line rather than a segment of a circumference.

In one embodiment, the imaging system 200 includes a control mechanism 208 to control movement of the components, such as the rotation of the gantry 102 and the operation of the X-ray radiation source 104. In certain embodiments, the control mechanism 208 further includes an X-ray controller 210, configured to provide power and timing signals to the X-ray radiation source 104. Additionally, the control mechanism 208 includes a gantry motor controller 212, configured to control the rotational speed and/or position of the gantry 102 on the basis of imaging requirements.

In certain embodiments, the control mechanism 208 further includes a data acquisition system (DAS) 214, configured to sample analog data received from the detector elements 202, and convert the analog data into digital signals for subsequent processing. The data sampled and digitized by the DAS 214 is transmitted to a computer or computing device 216. In one example, the computing device 216 stores data in a storage device 218. For example, the storage device 218 may include a hard disk drive, a floppy disk drive, a compact disc-read/write (CD-R/W) drive, a digital versatile disc (DVD) drive, a flash drive, and/or a solid-state storage drive.

Additionally, the computing device 216 provides commands and parameters to one or more of the DAS 214, the X-ray controller 210, and the gantry motor controller 212 to control system operations, such as data acquisition and/or processing. In certain embodiments, the computing device 216 controls system operations on the basis of operator input. The computing device 216 receives the operator input via an operator console 220 that is operably coupled to the computing device 216, the operator input including, for example, commands and/or scan parameters. The operator console 220 may include a keyboard (not shown) or a touch screen to allow the operator to specify commands and/or scan parameters.

Although FIG. 2 shows only one operator console 220, more than one operator console may be coupled to the imaging system 200, and, for example, is used to input or output system parameters, request examination, and/or view images. Moreover, in certain embodiments, the imaging system 200 may be coupled, via one or more configurable wired and/or wireless networks (such as the Internet and/or a virtual private network), to a plurality of displays, printers, workstations, and/or similar devices located locally or remotely within an institution or hospital or in a completely different location, for example.

In one embodiment, for example, the imaging system 200 includes a picture archiving and communication system (PACS) 224, or is coupled to the PACS. In one exemplary embodiment, the PACS 224 is further coupled to a remote system (such as a radiology information system or a hospital information system) and/or an internal or external network (not shown) to allow an operator at a different location to provide commands and parameters and/or acquire access to image data.

The computing device 216 uses operator-provided and/or system-defined commands and parameters to operate a workbench motor controller 226, which can in turn control a workbench motor 228. The workbench motor can adjust the position of the workbench 115 shown in FIG. 1. Specifically, the workbench motor controller 226 is moved in the workbench 115 via the workbench motor 228, so as to properly position the subject under examination 112 in the gantry 102 to acquire projection data corresponding to a target volume of the subject under examination 112. For example, the computing device 216 may send a command to the workbench motor controller 226, so as to instruct the workbench motor controller 226 to adjust, via the motor 228, the vertical position and/or the horizontal position of the workbench 115.

As described previously, the DAS 214 samples and digitizes the projection data acquired by the detector elements 202. Subsequently, an image reconstructor 230 uses the sampled and digitized X-ray data to perform high-speed reconstruction. Although the image reconstructor 230 is shown as a separate entity in FIG. 2, in certain embodiments, the image reconstructor 230 may form a part of the computing device 216. Alternatively, the image reconstructor 230 may not be present in the imaging system 200, and the computing device 216 may instead perform one or more functions of the image reconstructor 230. In addition, the image reconstructor 230 may be located locally or remotely and may be operably connected to the imaging system 200 by using a wired or wireless network. Specifically, in one exemplary embodiment, computing resources in a “cloud” network cluster may be used for the image reconstructor 230.

In one embodiment, the image reconstructor 230 stores a reconstructed image in the storage device 218. Alternatively, the image reconstructor 230 transmits a reconstructed image to the computing device 216 to generate usable patient information for diagnosis and evaluation. In certain embodiments, the computing device 216 transmits a reconstructed image and/or patient information to a display 232, the display being communicatively coupled to the computing device 216 and/or the image reconstructor 230. In one embodiment, the display 232 allows an operator to evaluate an imaged anatomical structure. The display 232 may further allow the operator to select a volume of interest (VOI) and/or request patient information by means of, for example, a graphical user interface (GUI) for subsequent scanning or processing.

As described further herein, the computing device 216 may include computer-readable instructions, and the computer-readable instructions are executable to send, according to an examination imaging scheme, commands and/or control parameters to one or more of the DAS 214, the X-ray controller 210, the gantry motor controller 212, and the workbench motor controller 226. The examination imaging scheme includes a clinical task/intent, also referred to herein as a clinical intent identifier (CID) of the examination. For example, the CID may indicate a goal (e.g., a general scan or lesion detection, an anatomical structure of interest, a quality parameter, or another goal) of the procedure on the basis of a clinical indication, and may further define the position and orientation (e.g., posture) of the subject under examination required during a scan (e.g., supine and feet first). The operator of the system 200 may then position the subject under examination on the workbench according to the position and orientation of the subject under examination specified by the imaging scheme. Further, the computing device 216 may set and/or adjust various scan parameters (e.g., a dose, a gantry rotation angle, kV, mA, and an attenuation filter) according to the imaging scheme. For example, the imaging scheme may be selected by the operator from a plurality of imaging schemes stored in a memory on the computing device 216 and/or a remote computing device, or the imaging scheme may be automatically selected by the computing device 216 according to received patient information.

During the examination/scanning phase, it may be desirable to expose the subject under examination to a radiation dose as low as possible while still maintaining the required image quality. In addition, reproducible and consistent imaging quality between examinations and between subjects under examination, as well as between different imaging system operators, may be required. Thus, an imaging system operator may manually adjust the position of the workbench and/or the position of the subject under examination, so as to, for example, center a desired anatomical structure of a patient at the center of a gantry bore. However, such a manual adjustment may be error-prone. Thus, the CID associated with the selected imaging scheme may be mapped to various positioning parameters of the subject under examination. The positioning parameters of the subject under examination include the posture and orientation of the subject under examination, the height of the workbench, an anatomical reference for scanning, and a starting and/or ending scan position.

Thus, the depth camera 114 may be operably and/or communicatively coupled to the computing device 216 to provide image data to determine the anatomy of the subject under examination, including the posture and orientation. Additionally, various methods and procedures described further herein for determining the patient anatomy on the basis of image data generated by the depth camera 114 may be stored as executable instructions in a non-transitory memory of the computing device 216.

Additionally, in some examples, the computing device 216 may include a camera image data processor 215 that includes instructions for processing information received from the depth camera 114. The information (which may include depth information and/or visible light information) received from the depth camera 114 may be processed to determine various parameters of the subject under examination, such as the identity of the subject under examination, the physique (e.g., the height, weight, and patient thickness) of the subject under examination, and the current position of the subject under examination relative to the workbench and the depth camera 114. For example, prior to imaging, the body contour or anatomy of the subject under examination 112 may be estimated by using images reconstructed from point cloud data, the point cloud data being generated by the camera image data processor 215 according to images received from the depth camera 114. The computing device 216 may use these parameters of the subject under examination to perform, for example, patient-scanner contact prediction, scan range superposition, and scan key point calibration, as will be described in further detail herein. Further, data from the depth camera 114 may be displayed via the display 232.

In one embodiment, information from the depth camera 114 may be used by the camera image data processor 215 to track one or more subjects under examination in the field of view of the depth camera 114. In some examples, skeleton tracking may be performed by using image information (e.g., depth information), where a plurality of joints of the subject under examination are identified and analyzed to determine the motion, posture, position, and so on of the subject under examination. The positions of joints during the skeleton tracking can be used to determine the above-described parameters of the subject under examination. In other examples, the image information may be directly used to determine the above-described parameters of the subject under examination without skeleton tracking.

On the basis of these positioning parameters of the subject under examination, the computing device 216 may output one or more alerts to the operator regarding the posture/orientation of the patient and the prediction of examination (e.g., scan) results, thereby reducing the possibility of the subject under examination being exposed to a higher than desired radiation dose, and improving the quality and reproducibility of the image generated by the scan. As an example, the estimated body structure may be used to determine whether the subject under examination is in an imaging position specified by the radiologist, thereby reducing the incidence of repeating the scan due to improper positioning. Furthermore, the amount of time an imaging system operator spends positioning the subject under examination can be reduced, allowing more scans to be performed per day and/or allowing additional interaction with the subject under examination.

A plurality of exemplary patient orientations may be determined on the basis of data received from a depth camera (such as the depth camera 114 described in FIG. 1 and FIG. 2). For example, a controller (e.g., the computing device 216 in FIG. 2) may perform patient anatomy extraction and posture estimation on the basis of images received from the depth camera, thereby enabling different patient orientations to be distinguished from each other.

The CT imaging system 100 may perform imaging examination on the basis of a scanning protocol. The scanning protocol is a description of the imaging examination. The scanning protocol may include a description of an involved body part, for example, a medical or colloquial term for the body part. The scanning protocol may provide various parameters and related information for performing scans and post-processing, such as a power value, the duration of radiation, speed of movement, radiation energy, and a time delay between image captures, etc. It is conceivable that any configurable technical parameter that should be used for imaging examination by the imaging system 110 may be defined in the scanning protocol.

The CT imaging system 100 may have an automatic patient positioning function. That is, a patient may be automatically positioned at a scan start position in an opening of the gantry 102 on the basis of an examination instruction or the scanning protocol, and moved in the longitudinal direction to a scan end position during scanning and imaging. The current automatic patient positioning function may automatically determine a scan range in the longitudinal direction on the basis of an anatomical structure to be imaged (e.g., from an examination instruction or the scanning protocol) and the patient anatomy from the depth camera 114. However, since the accuracy of the body contour acquired by the depth camera 114 is limited and since automatic positioning is substantially for an average body contour center of all scout scan ranges, the accuracy of centering a scan subject at the isocenter of a medical imaging device needs to be improved.

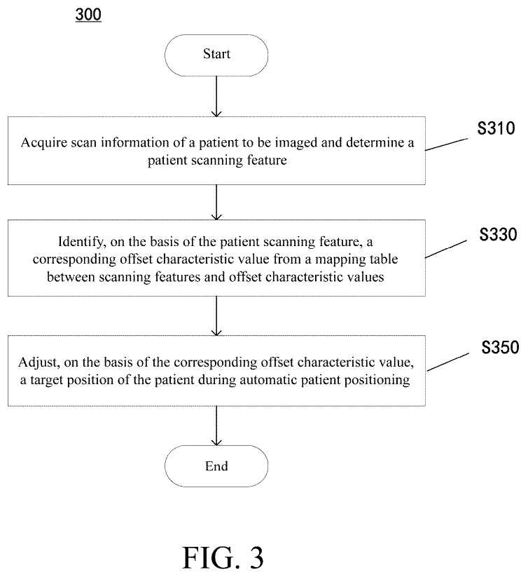

According to an embodiment of the present invention, a method for automatic patient positioning in medical imaging is provided. Reference is made to FIG. 3, which is a flowchart of a method 300 for automatic patient positioning according to an exemplary embodiment of the present invention. In the embodiment, the method 300 is applied to, for example, the imaging system 100 or 200 shown in FIG. 1 or FIG. 2. As shown in FIG. 3, the method 300 for automatic patient positioning according to the embodiment may include the following steps S310 to S350.

Step S310 is as follows: acquiring scan information of a patient to be imaged and determining a patient scanning feature.

The scan information may be part or all of information derived from an examination instruction or a scanning protocol. The examination instruction may come from a radiology information system (RIS), may be selected from a predefined list stored on the medical imaging system 100, or may be input by a user by using an input unit (e.g., via a keyboard or voice). The examination instruction may include, for example, the age, sex, weight, body part (such as the kidney, liver, spleen, stomach, adrenal glands, pancreas, colon, or the like), or body region (such as the brain, neck, heart, chest, abdomen, knee, or the like) of the patient, previous diagnosis, the type of the imaging device, medical institution-specific applications of the imaging device, layout planning of the imaging device, and/or contrast agent information. It would be conceivable for those skilled in the art that the examination instruction may further include other parameters. The scanning protocol may be pre-stored in the medical imaging system 100, for example, in a memory of the computing device 216. The scanning protocol may also be stored in a separate memory or in a remote cloud, and in this case, the scanning protocol may be transmitted to the computing device 216 via a wired or wireless network. The scanning protocol may be provided by the manufacturer of the imaging system 110, or may be predefined by the user of the medical imaging system 100. The scanning protocol may also be provided by users of medical imaging systems of other medical institutions. In an embodiment of the present application, the scan information includes at least information about the body part or body region to be imaged in order to facilitate determination of the patient scanning feature. For example, the scan information may include a scanning protocol and/or a scanning descriptor, and the step of determining a patient scanning feature may include: determining, on the basis of the scanning protocol and/or the scanning descriptor, an anatomical structure category of the patient to be imaged. The scanning descriptor may include specific terms extracted from the examination instruction.

Step S330 is as follows: identifying, on the basis of the patient scanning feature, a corresponding offset characteristic value from a mapping table between scanning features and offset characteristic values.

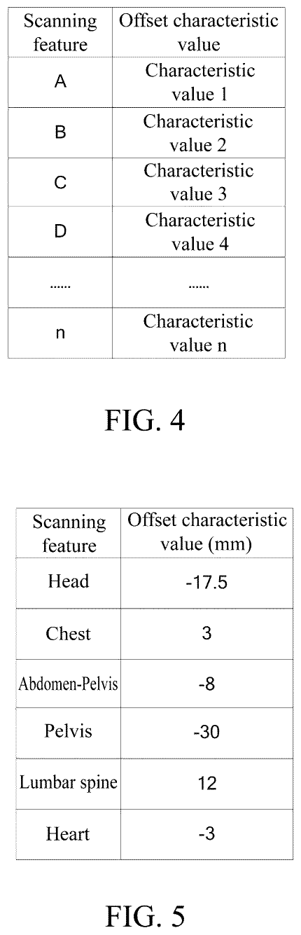

FIG. 4 shows a schematic diagram of a mapping table between scanning features and offset characteristic values. For example, if it is determined in step S310 that the patient scanning feature is C, then in step S330, the corresponding offset characteristic value (i.e., characteristic value 3) can be identified from the mapping table on the basis of scanning feature C.

In some embodiments of the present invention, the scanning feature may include an anatomical structure category, such as the head, chest, abdomen-pelvis, pelvis, lumbar spine, and heart. FIG. 5 shows an example of a mapping table. As shown, different anatomical structure categories and associated characteristic values (for example, in millimeters) are stored in the mapping table. It is noted that the present invention is not intended to limit the number and characteristics of the anatomical structure categories, and those skilled in the art could conceive of different combinations of anatomical structure categories.

Step S350 is as follows: adjusting, on the basis of the corresponding offset characteristic value, a target position of the patient in the automatic patient positioning. For example, the centering in the automatic patient positioning may be based on an average body contour center of the patient estimated by a 3D depth camera, and then the average body contour center is aligned with an isocenter of the CT system, so as to configure the position of the patient in a vertical direction (a Y direction of the medical imaging system) and/or a transverse direction (an X direction of the medical imaging system). In this way, in step S350, the position of the patient in the vertical direction or the transverse direction may be modified by using the corresponding offset characteristic value, thereby compensating for a deviation between the center of the anatomical structure to be imaged and the isocenter of the CT system.

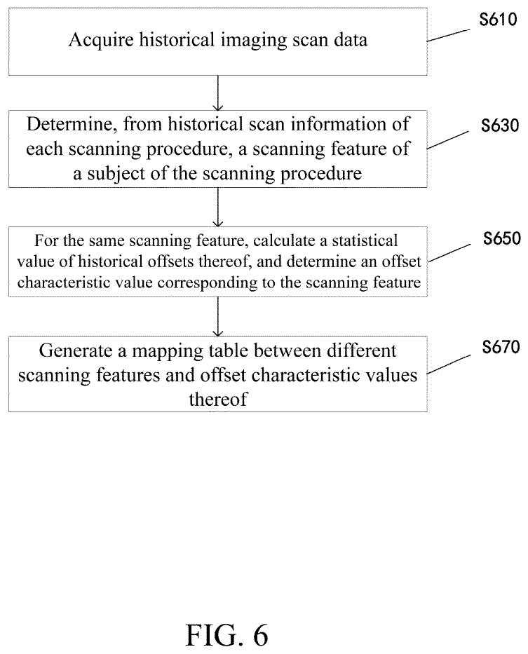

Some embodiments of the present invention, the mapping table may be established via steps S610 to S670, as shown in FIG. 6.

Referring to FIG. 6, step S610 is as follows: acquiring historical imaging scan data. The historical imaging scan data may include historical scan information and historical offsets associated with a plurality of scanning procedures. In some preferred embodiments of the present invention, the plurality of scanning procedures may be for the same medical imaging system or the same type of medical imaging systems, and may employ the same automatic patient positioning function under substantially the same environmental conditions. Substantially the same environmental conditions may be substantially the same on-site conditions of the medical imaging system and substantially the same camera mounting position. The same automatic patient positioning function may be automatic patient positioning employing the same algorithm on the basis of a 3D depth camera. The historical scan information may include a scanning protocol and/or a scanning descriptor, as described above.

Step S630 is as follows: determining, from historical scan information of each scanning procedure, a scanning feature of a subject of the scanning procedure. The scanning feature of the subject includes an anatomical structure category of the subject. The anatomical structure category of the subject may be determined on the basis of the scanning protocol and/or the scanning descriptor.

Step S650 is as follows: for the same scanning feature, calculating a statistical value of historical offsets thereof, and determining an offset characteristic value corresponding to the scanning feature. The statistical value of historical offsets may be an average value of a plurality of historical offsets, but the present invention is not limited thereto, and other statistical values such as a weighted average value, a median value, and the like are conceivable. In some embodiments of the present invention, the statistical value (A) of historical offsets may be determined as the offset characteristic value. In some other embodiments of the present invention, the inverse (−A) of the statistical value of historical offsets may be determined as the offset characteristic value.

Step S670 is as follows: generating a mapping table between different scanning features and offset characteristic values thereof.

In some embodiments of the present invention, the scanning feature may further include an age range, sex, and/or a body type of the subject, so that the historical scan information may include information for determining the age range, sex, and/or body type of the subject. The information for determining the body type of the patient may include the BMI of the patient, or the body contour of the patient acquired by using a 3D camera.

In some embodiments of the present invention, the historical scan information may further include a positioning identifier, used for identifying whether manual positioning (identifier “1”) or automatic positioning (identifier “0”) is used in each scanning procedure. In this way, the mapping table may be generated only for scanning procedures using automatic positioning among the plurality of scanning procedures. The positioning identifier may be extracted from a log file of the automatic patient positioning.

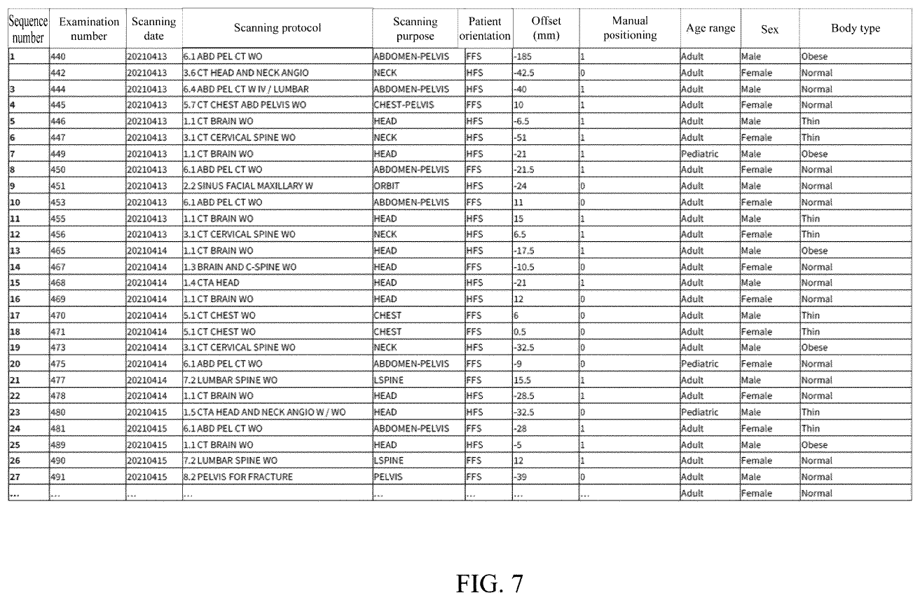

FIG. 7 shows a screenshot of an exemplary table of historical imaging scan data acquired during establishment of a mapping table. In this example, the historical imaging scan data includes, for each examination, a protocol description, a scan purpose (which may be used as the scanning descriptor), a patient orientation (head first or feet first), a positioning identifier (1 indicates using manual positioning, and 0 indicates not using manual positioning), an age range, sex, a body type, etc. The historical imaging scan data further includes an offset for each examination (a vertical or transverse deviation between the center of the anatomical structure and the isocenter of the CT system in each imaging).

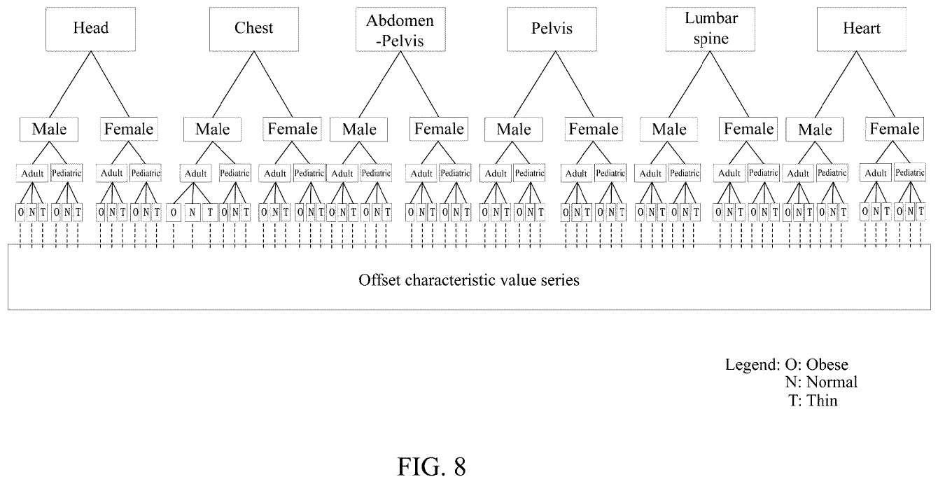

In some embodiments of the present invention, a mapping table may be established for a plurality of scanning features. For example, the anatomical structure category, sex, age range, and body type may be selected from the example in FIG. 7 to establish a multi-level mapping table. As shown in FIG. 8, a mapping table established based on six anatomical structure categories (head, chest, abdomen-pelvis, pelvis, lumbar spine, and heart), sex (male and female), age range (adult and pediatric), and body type (obese O, normal N, and thin T) may include 6*2*2*3=72 mapping entries. The same anatomical structure category, sex, age range, and body type correspond to one offset characteristic value, and said offset characteristic value may be a statistical value (e.g., an average value) of a plurality of offset values corresponding to a plurality of scanning procedures having the same anatomical structure category, sex, age range, and body type in the historical scan information. Furthermore, it is possible to only select those scanning procedures that do not use manual positioning from the historical scan information, so as to calculate the offset statistical value. In some cases, the automatic patient positioning operation may result in a large deviation in the vertical direction or the transverse direction, and an operator of the imaging system may choose to correct such a deviation via manual adjustment. The offset value data of the scanning procedures including manual adjustment needs to be excluded to avoid affecting the objectivity of the statistical value of the offset value data resulting from the automatic patient positioning. In some embodiments of the present invention, for the same scanning feature (e.g., the same anatomical structure category, sex, age range, and body type shown in FIG. 8), more than a threshold number of scanning procedures may be selected to establish the mapping table, and the threshold number may be 10, 20, 30, 40, or greater. In this way, offset statistical values with optimized accuracy will be generated.

In some embodiments of the present invention, the offset in the historical scan information may be acquired by processing an original medical image. In various embodiments, original medical image data may be stored according to the DICOM standard or data model, so as to obtain a DICOM image. A DICOM file may include a file header portion, a file meta information portion, and a single service object pair (SOP) instance. The file header portion includes a 128-byte preamble followed by characters DICM, all in uppercase. The file header portion is followed by the file meta information portion. This portion follows a markup file format, and includes information or metadata about the file, such as a patient corresponding thereto (e.g., patient name, identification number of the patient, birth date of the patient, etc.), a study corresponding thereto (e.g., universal identifier (UID) of the study, date of the study, consulting physician, accession number, etc.), series and study corresponding thereto (e.g., series UID, series number, modality type, etc.), and so on. The metadata allows DICOM field querying and indexing.

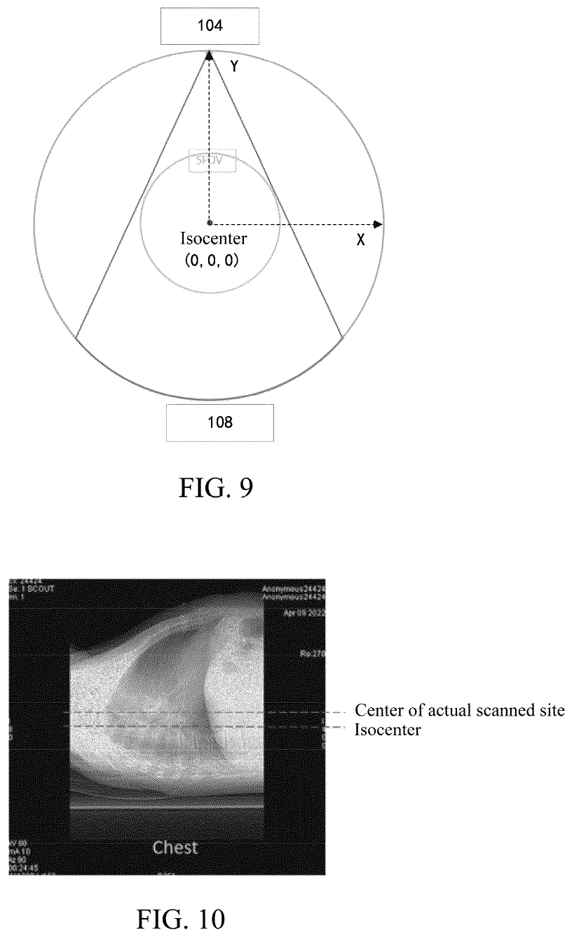

Referring to FIG. 9, FIG. 9 shows a schematic diagram of a scan field of view (SFOV) of an X-ray fan beam or cone beam projected by the X-ray radiation source 104 to the detector array 108. The CT system has its own coordinate system. The center of the scan field of view (SFOV) is the isocenter of the CT system, and the default coordinates are (0, 0, 0), which is the optimal center of positioning. After a patient is positioned, an offset between the center of an actually scanned site and the isocenter actually represents an error in the Y direction or the X direction. FIG. 10 shows an example of an error in the Y direction. In a Z direction (i.e., the direction in which the patient enters and exits the gantry opening) perpendicular to the XY plane, the coordinate of each point in the Y direction or the X direction is recorded as a vector in the DICOM header portion. Thus, the offset of each scanning procedure may be determined in the following manner: acquiring an original DICOM file of each scanning procedure, and reading a coordinate vector in a DICOM header portion; removing air and a scanning table portion according to the magnitude of a CT value; starting from the minimum coordinate vector of a scanned site, only extracting the middle 30% to 70% of coordinate vectors of the scanned site; and acquiring an average value of the extracted coordinate vectors to acquire an actual DICOM center value (equivalent to the center of the actually scanned site).

In some embodiments of the present invention, the offset in the Y direction may be determined for the same scanning feature (e.g., the same anatomical structure category, sex, age range, and body type shown in FIG. 8) on the basis of a DICOM file obtained by a 90° scout scan (FIG. 10 shows an image example of the 90° scout scan). Similarly, the offset in the X direction may be determined for the same scanning feature on the basis of a scout scan at another angle.

In some embodiments of the present invention, when the scan information includes a preset scan range, the anatomical structure category in the patient scanning feature may further be determined in step S310 in the following manner: identifying a plurality of landmarks of the patient by using a 3D camera, and determining an anatomical structure category of the patient on the basis of a positional relationship between at least one of the plurality of landmarks and the scan range.

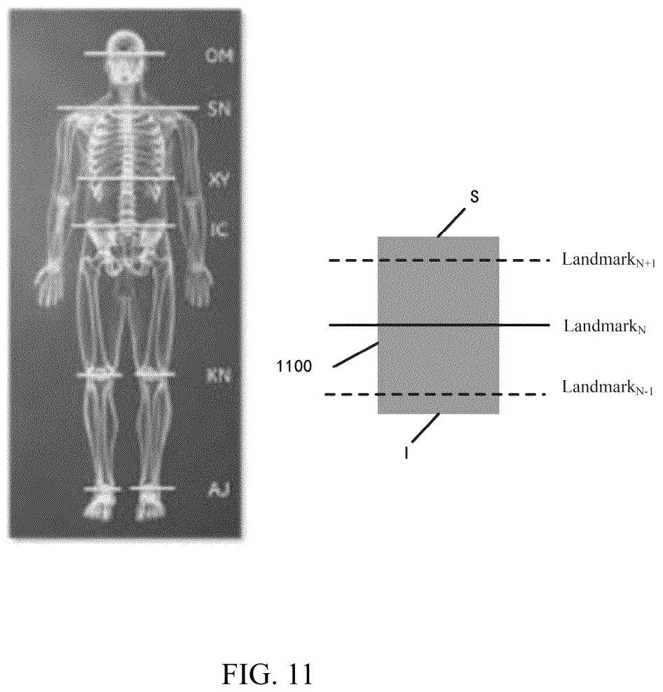

Referring to FIG. 11, the left side of the drawing shows an example of human body landmarks. The right side shows a schematic diagram of a positional relationship between a start position (S) and an end position (I) of a scan range 1100 in a viewing angle of a 3D camera and a landmark (Landmark_{N}) and preceding and following landmarks thereof (Landmark_{N+1} and Landmark_{N-1}). In the schematic diagram, the scan range 1100 covers the landmark (Landmark_{N}) and the preceding and following landmarks thereof ((Landmark_{N+1} and Landmark_{N-1}), which may be expressed as S>Landmark_{N+1}>Landmark_{N}>Landmark_{N-1}>I.

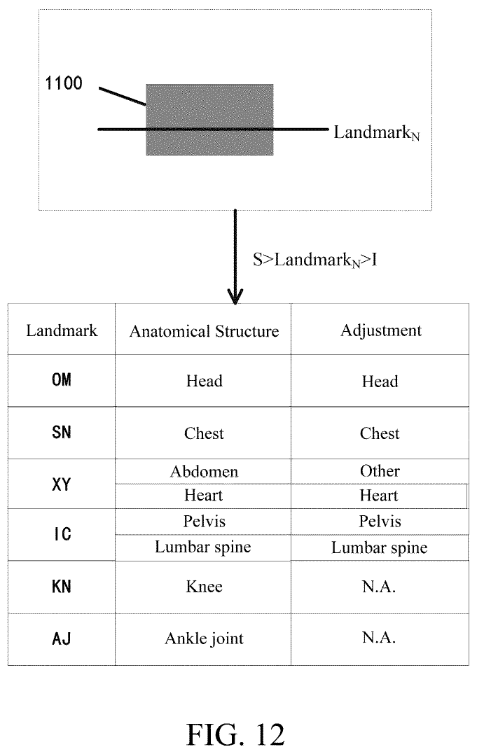

Schematic diagrams showing the principle of determining the anatomical structure category of the patient according to the positional relationship between at least one landmark and the scan range 1100 are described below with reference to FIGS. 12-16.

FIG. 12 shows the case in which the scan range 1100 covers a landmark (Landmark_{N}) (S>Landmark_{N}>I). Assuming that the landmark (Landmark_{N}) is each of the six exemplary landmarks shown on the left side of FIG. 11, the anatomical structure category of the patient can be determined with reference to the correspondence table in FIG. 12. Specifically, when the landmark (Landmark_{N}) is OM, the anatomical structure category of the patient can be determined as the head, so that the corresponding offset characteristic value can subsequently be identified from a mapping table between scanning features including the head and the offset characteristic values, so as to make an adjustment. When the landmark (Landmark_{N}) is SN, the anatomical structure category of the patient can be determined as the chest, so that the corresponding offset characteristic value can subsequently be identified from a mapping table between scanning features including the chest and the offset characteristic values, so as to make an adjustment. When the landmark (Landmark_{N}) is XY, the anatomical structure category of the patient may be the abdomen or the heart. In this case, a determination needs to be made with reference to other scan information. For example, the examination instruction or the scanning protocol may be searched for the keyword “heart” to determine whether the anatomical structure category is the heart. If so, the corresponding offset characteristic value may subsequently be identified from a mapping table between scanning features including the heart and the offset characteristic values, so as to make an adjustment. If not, the anatomical structure category of the patient may be the abdomen. In this case, automatic centering for the body may be considered to be sufficient for an abdominal scan, and therefore no adjustment is made (indicated as “other” in the drawing). When the landmark (Landmark_{N}) is IC, the anatomical structure category of the patient may be the pelvis or the lumbar. In this case, a determination needs to be made with reference to other scan information. For example, the examination instruction or the scanning protocol may be searched for the keyword “lumbar” to determine whether the anatomical structure category is the lumbar. If so, the corresponding offset characteristic value may subsequently be identified from a mapping table between scanning features including the lumbar and the offset characteristic values, so as to make an adjustment. If not, the anatomical structure category of the patient may be the pelvis, and the corresponding offset characteristic value may subsequently be identified from a mapping table between scanning features including the pelvis and the offset characteristic values, so as to make an adjustment. When the landmark (Landmark_{N}) is KN or AJ, the anatomical structure category of the patient can be determined as the knee or the ankle joint.

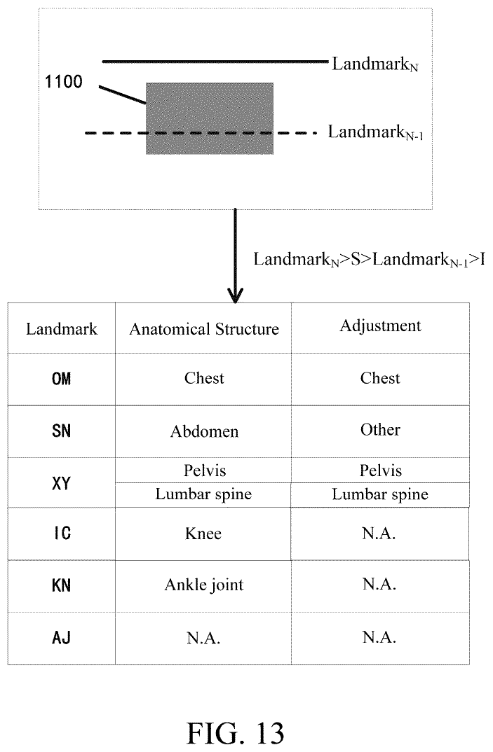

FIG. 13 shows the case in which Landmark_{N}>S>Landmark_{N-1}>I. Assuming that the landmark (Landmark_{N}) is each of the six exemplary landmarks shown on the left side of FIG. 11, the anatomical structure category of the patient can be determined with reference to the correspondence table in FIG. 13. Some details of the determination of the anatomical structure category in FIG. 13 are the same as or similar to those in FIG. 12, and will not be described herein again.

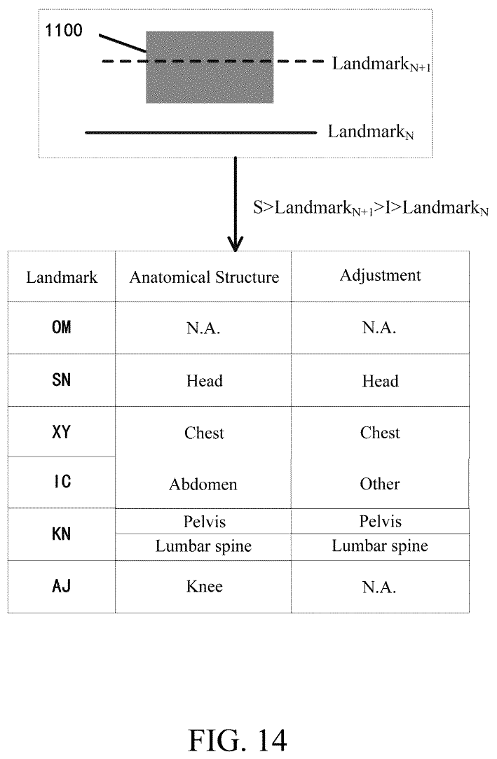

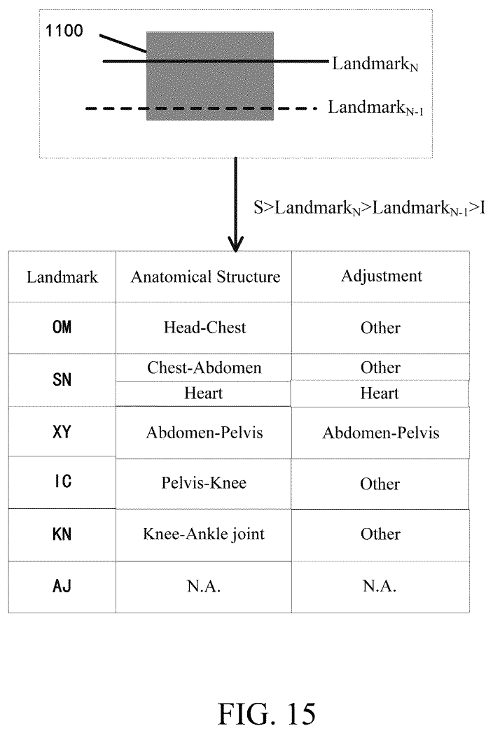

FIG. 14 shows the case in which S>Landmark_{N+1}>I>Landmark_{N}. Assuming that the landmark (Landmark_{N}) is each of the six exemplary landmarks shown on the left side of FIG. 11, the anatomical structure category of the patient can be determined with reference to the correspondence table in FIG. 14. Some details of the determination of the anatomical structure category in FIG. 14 are the same as or similar to those in FIG. 12, and will not be described herein again.

FIG. 15 shows the case in which S>Landmark_{N}>Landmark_{N-1}>I. Assuming that

the landmark (Landmark_{N}) is each of the six exemplary landmarks shown on the left side of FIG.

11, the anatomical structure category of the patient can be determined with reference to the correspondence table in FIG. 15. Some details of the determination of the anatomical structure category in FIG. 15 are the same as or similar to those in FIG. 12, and will not be described herein again.

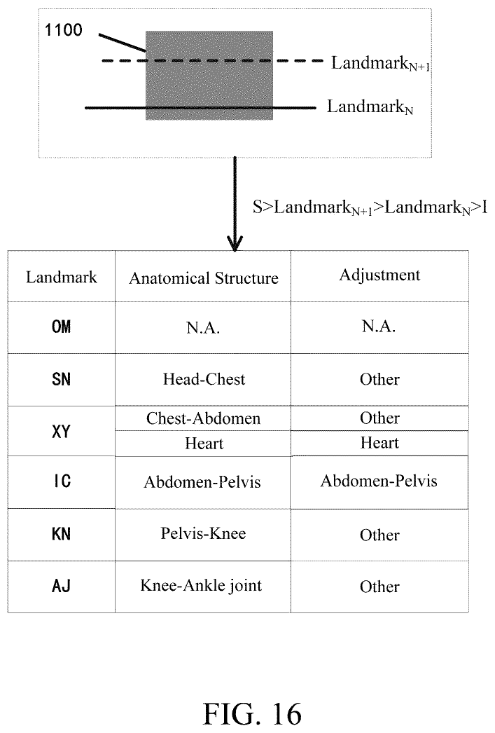

FIG. 16 shows the case in which S>Landmark_{N+1}>Landmark_{N}>I. Assuming that the landmark (Landmark_{N}) is each of the six exemplary landmarks shown on the left side of FIG. 11, the anatomical structure category of the patient can be determined with reference to the correspondence table in FIG. 16. Some details of the determination of the anatomical structure category in FIG. 16 are the same as or similar to those in FIG. 12, and will not be described herein again.

In some embodiments of the present invention, the method 300 may further include adaptive optimization of the mapping table. For example, after each examination, scanning, and imaging performed by the medical imaging system, a DICOM image that uses automatic patient positioning instead of manual positioning may be processed to acquire a new offset, and a corresponding entry in the mapping table is updated on the basis of the new offset. Alternatively, adaptive optimization may also be performed on a regular cycle. For example, after examination, scanning, and imaging is performed by the medical imaging system several times (e.g., 40 times), DICOM images from these 40 times are processed to acquire new offset statistical values for different scanning features, and then offset statistical values in the mapping table are corrected on that basis to update the mapping table.

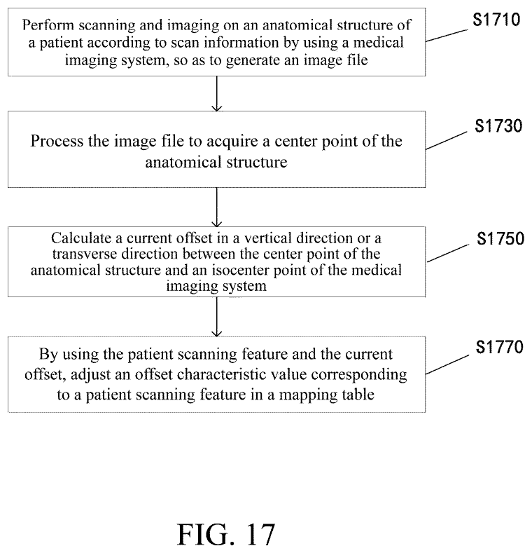

Thus, referring to FIG. 17, adaptive optimization of the mapping table may be implemented via steps S1710 to S1770. Step S1710 is as follows: performing scanning and imaging on an anatomical structure of the patient according to the scan information by using the medical imaging system 100, so as to generate an image file. During the scanning and imaging, the patient is moved in the longitudinal direction (i.e., the Z direction of the medical imaging system, which is a direction perpendicular to the XY plane). For example, the image file may be a DICOM file.

Step S1730 is as follows: processing the image file to acquire a center point of the anatomical structure. The acquisition of the center point may be implemented in the manner described with reference to FIG. 9 and FIG. 10.

Step S1750 is as follows: calculating a current offset in a vertical direction or a transverse direction between the center point of the anatomical structure and an isocenter point of the medical imaging system. In some embodiments, the isocenter point may have default coordinates (0, 0, 0), and in this case, the Y coordinate or the X coordinate of the center point of the anatomical structure represents the offset.

Step S1770 is as follows: by using the patient scanning feature and the current offset, adjusting the offset characteristic value corresponding to the patient scanning feature in the mapping table. The adjustment may be made by performing statistical computation on the offset characteristic value corresponding to the patient scanning feature and the current offset, and by determining a new offset characteristic value. In some embodiments, if the offset characteristic value is a statistical value of historical offsets, then the statistical computation may be a summation. In some other embodiments, if the offset characteristic value is the inverse of the statistical value of historical offsets, the statistical computation may be a difference calculation.

In some embodiments of the present invention, if the patient scanning feature includes the body type of the patient, the information for determining the body type of the patient may include BMI of the patient, or a body contour of the patient acquired by using a 3D camera. In other words, the information for determining the body type of the patient may acquire the BMI of the patient from a radiology information system (RIS). Alternatively, if BMI data of the patient is missing, the body contour of the patient acquired by the 3D depth camera 114 may be employed, and is used to analyze and evaluate the body type of the patient.

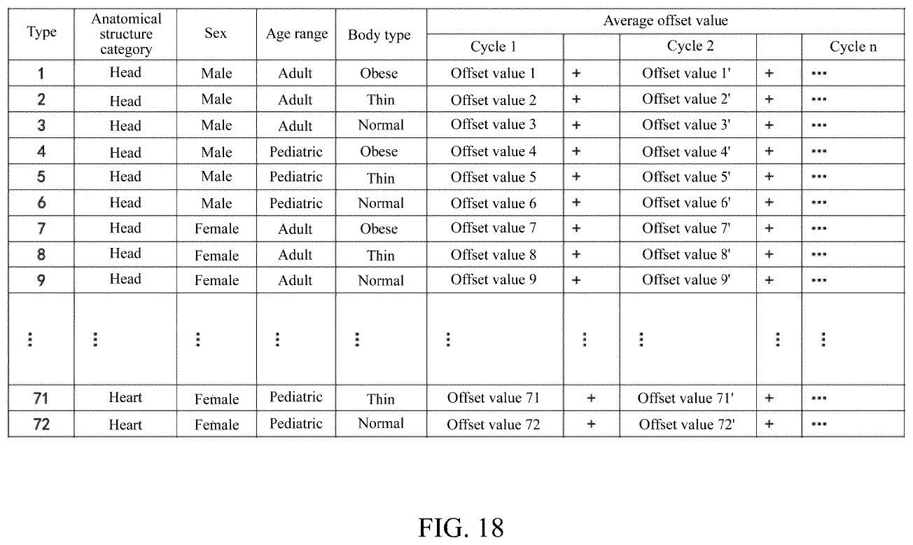

Referring to FIG. 18, FIG. 18 exemplarily shows a schematic diagram of a mapping table generated according to the method described above and adaptive optimization. In the example, the patient scanning features include anatomical structure categories (for example, six categories), sex (male/female), age range (adult/pediatric), and body type (obese/thin/normal), totally 72 combinations. Therefore, the mapping table has entries representing 72 mapping relationships. A plurality of scanning procedures are performed during cycle 1. When valid data (using automatic patient positioning without manual intervention-related offsets) of each combination reaches a certain amount (e.g., 40 times), the average offset values (i.e., offset value 1, offset value 2, . . . , and offset value 72) can be calculated. In this way, the centering effect can be optimized on the basis of the mapping table in subsequent scans performed by the imaging system 100. Then, after optimization has been performed using the mapping table for a period of time (i.e., cycle 2), for a plurality of scanning procedures in cycle 2, when valid data (using automatic patient positioning without manual intervention-related offsets) of each combination reaches a certain amount (e.g., 1, 10, 20, 40 or more times), the average offset values (i.e., offset value 1′, offset value 2′, . . . , and offset value 72′) that are still present in the automatic patient positioning even after optimization has been performed using the mapping table can be calculated. In this way, the average offset values in the mapping table may be updated by respectively adding offset value 1, offset value 2, . . . , and offset value 72 of cycle 1 and offset value 1′, offset value 2′, . . . , and offset value 72′ of cycle 2, thereby achieving adaptive optimization of the mapping table. The adaptive optimization of the mapping table may be implemented on the basis of periodic generation and iteration of the average offset value, thereby constantly improving optimization of the centering effect for automatic patient positioning using the method of the present application.

Similar to the above method, a corresponding medical imaging system is further provided in the present invention.

The medical imaging system (e.g., the imaging system 100 or 200 in FIG. 1 or 2) may include a medical imaging apparatus configured to perform scanning and imaging on a patient, a patient moving table, a 3D depth camera, an automatic positioning module, and a positioning optimization module.

The patient moving table (e.g., the workbench 115) is configured to support the patient, and is movable in a horizontal direction (including a transverse direction and a longitudinal direction) and a vertical direction. During scanning and imaging, the patient moving table is moved in the longitudinal direction (perpendicular to a ray beam plane). The 3D depth camera (e.g., the depth camera 114) is configured to acquire a body contour of the patient. The automatic positioning module is configured to perform automatic positioning of the patient according to scan information of the patient and the acquired body contour. The positioning optimization module is configured to perform the following operations: acquiring scan information and determining a patient scanning feature; identifying, on the basis of the patient scanning feature, a corresponding offset characteristic value from a mapping table between scanning features and offset characteristic values; and adjusting, on the basis of the corresponding offset characteristic value, a target position of the patient in the automatic positioning. The medical imaging apparatus may be configured to perform scanning and imaging on an anatomical structure of the patient according to the scan information, so as to generate an image file. The medical imaging system may further include an adjustment module configured to: process the image file to acquire a center point of the anatomical structure; calculate a current offset in a vertical direction or a transverse direction between the center point of the anatomical structure and an isocenter point of the medical imaging system; and by using the patient scanning feature and the current offset, adjust the offset characteristic value corresponding to the patient scanning feature in the mapping table. Operation details of the positioning optimization module and the adjustment module are the same as or similar to those in the method 300 described above, and will not be described herein again.

One or a plurality of the above-described techniques and/or embodiments may be implemented using hardware and/or software or include hardware and/or software, for example, modules or apparatuses executed on one or a plurality of computing devices 216. Of course, the modules or apparatuses described herein show various functions and are not limited to limiting the structure and functions of any embodiment. On the contrary, the functions of various modules or apparatuses may be divided and executed differently according to more or fewer modules or apparatuses considered by various designs.

### Exemplary Computing Device

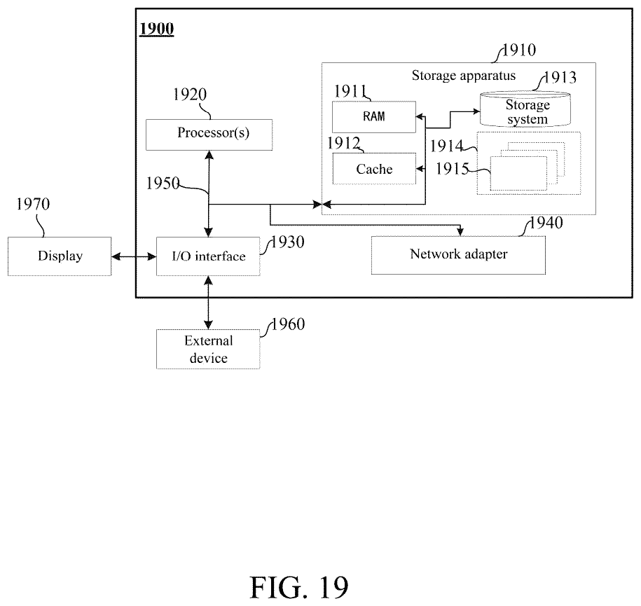

FIG. 19 shows an example of an electronic device 1900 according to an embodiment of the present invention. The electronic device 1900 includes: one or a plurality of processors 1920; and a storage apparatus 1910 configured to store one or a plurality of programs, wherein when the one or plurality of programs are executed by the one or plurality of processors 1920, the one or plurality of processors 1920 are caused to implement the method for optimizing automatic patient positioning provided in the embodiments of the present invention. The processor is, for example, a digital signal processor (DSP), a microcontroller, an application-specific integrated circuit (ASIC), or a microprocessor.

The electronic device 1900 shown in FIG. 19 is only an example, and should not impose any limitation on the function and application scope of the embodiments of the present invention.

As shown in FIG. 19, the electronic device 1900 is represented in the form of a general-purpose computing device. Components of the electronic device 1900 may include, but are not limited to: one or a plurality of processors 1920, a storage apparatus 1910, and a bus 1950 connecting different system components (including the storage apparatus 1910 and the processor 1920).

The bus 1950 represents one or a plurality of types of bus structures, including a memory bus or a memory controller, a peripheral bus, an accelerated graphics port, a processor, or a local bus using any bus structure among the plurality of bus structures. For example, these architectures include, but are not limited to, an industrial standard architecture (ISA) bus, a micro channel architecture (MAC) bus, an enhanced ISA bus, a video electronics standards association (VESA) local bus, and a peripheral component interconnect (PCI) bus.

The electronic device 1900 typically includes a variety of computer system readable media. These media may be any available media that can be accessed by the electronic device 1900, including volatile and non-volatile media as well as removable and non-removable media.

The storage apparatus 1910 may include a computer system readable medium in the form of a volatile memory, for example, a random access memory (RAM) 1911 and/or a cache memory 1912. The electronic device 1900 may further include other removable/non-removable, and volatile/non-volatile computer system storage media. For example only, the storage system 1913 may be configured to read and write a non-removable non-volatile magnetic medium (which is not shown in FIG. 19, and is generally referred to as a “hard drive”). Although not shown in FIG. 19, a magnetic disk drive for reading and writing a removable non-volatile magnetic disk (such as a “floppy disk”) and an optical disc drive for reading and writing a removable non-volatile optical disc (such as a CD-ROM, a DVD-ROM, or other optical media) may be provided. In these cases, each drive may be connected to the bus 1950 via one or a plurality of data medium interfaces. The storage apparatus 1910 may include at least one program product, the program product has a group (for example, at least one) of program modules, and these program modules are configured to perform the functions of the various embodiments in the present invention.

A program/utility tool 1914 having a group (at least one) of program modules 1915 may be stored in, for example, the storage apparatus 1910. This program module 1915 includes, but is not limited to, an operating system, one or a plurality of application programs, other program modules, and program data, and each of these examples or a certain combination thereof may include implementation of a network environment. The program module 1915 typically performs the function and/or method in any embodiment described in the present invention.

The electronic device 1900 may also communicate with one or a plurality of peripheral devices 1960 (such as a keyboard, a pointing device, and a display 1970), and may also communicate with one or a plurality of devices that enable a user to interact with the electronic device 1900, and/or communicate with any device (such as a network card and a modem) that enables the electronic device 1900 to communicate with one or a plurality of other computing devices. Such communication may be performed via an input/output (I/O) interface 1930. In addition, the electronic device 1900 may also communicate with one or a plurality of networks (for example, a local area network (LAN), a wide area network (WAN), and/or a public network, such as the Internet) via a network adapter 1940. As shown in FIG. 19, the network adapter 1940 communicates with other modules of the electronic device 1900 through the bus 1950. It should be understood that although not shown in the drawing, other hardware and/or software modules may be used in conjunction with the electronic apparatus 1900, the modules including, but not limited to: microcode, device drivers, redundant processing units, external disk drive arrays, RAID systems, tape drives, data backup storage systems, and the like.

The processor 1920, by running programs stored in the storage apparatus 1910, executes various functional applications and data processing, such as implementing a video processing method provided by an embodiment of the present invention.

The technique described herein may be implemented with hardware, software, firmware, or any combination thereof, unless specifically described as being implemented in a specific manner. Any features described as modules or components may also be implemented together in an integrated logical apparatus, or separately implemented as discrete but interoperable logical apparatuses. If implemented with software, the technique may be implemented at least in part by a non-transitory processor-readable storage medium that includes instructions, where when executed, the instructions perform one or more of the aforementioned methods. The non-transitory processor-readable data storage medium may form part of a computer program product that may include an encapsulation material. Program code may be implemented in a high-level procedural programming language or an object-oriented programming language so as to communicate with a processing system. If desired, the program code may also be implemented in an assembly language or a machine language. In fact, the mechanisms described herein are not limited to the scope of any particular programming language. In any case, the language may be a compiled language or an interpreted language.

One or a plurality of aspects of at least some embodiments may be implemented by representative instructions that are stored in a machine-readable medium and represent various logic in a processor, where when read by a machine, the representative instructions cause the machine to manufacture the logic for executing the technique described herein.

Such machine-readable storage media may include, but are not limited to, a non-transitory tangible arrangement of an article manufactured or formed by a machine or device, including storage media, such as: a hard disk; any other types of disk, including a floppy disk, an optical disk, a compact disk read-only memory (CD-ROM), compact disk rewritable (CD-RW), and a magneto-optical disk; a semiconductor device such as a read-only memory (ROM), a random access memory (RAM) such as a dynamic random access memory (DRAM) and a static random access memory (SRAM), an erasable programmable read-only memory (EPROM), a flash memory, and an electrically erasable programmable read-only memory (EEPROM); a phase change memory (PCM); a magnetic or optical card; or any other type of medium suitable for storing electronic instructions.

Instructions may further be sent or received via a network interface device that uses any of a number of transport protocols (for example, Frame Relay, Internet Protocol (IP), Transfer Control Protocol (TCP), User Datagram Protocol (UDP), and Hypertext Transfer Protocol (HTTP)) and through a communication network using a transmission medium.

An exemplary communication network may include a local area network (LAN), a wide area network (WAN), a packet data network (for example, the Internet), a mobile phone network (for example, a cellular network), a plain old telephone service (POTS) network, and a wireless data network (for example, Institute of Electrical and Electronics Engineers (IEEE) 802.11 standards referred to as Wi-Fi®, and IEEE 802.19 standards referred to as WiMax®), IEEE 802.15.4 standards, a peer-to-peer (P2P) network, and the like. In an example, the network interface device may include one or a plurality of physical jacks (for example, Ethernet, coaxial, or phone jacks) or one or a plurality of antennas for connection to the communication network. In an example, the network interface device may include a plurality of antennas that wirelessly communicate using at least one technique of single-input multiple-output (SIMO), multiple-input multiple-output (MIMO), or multiple-input single-output (MISO) techniques.

The term “transmission medium” should be considered to include any intangible medium capable of storing, encoding, or carrying instructions for execution by a machine, and the “transmission medium” includes digital or analog communication signals or any other intangible medium for facilitating communication of such software.

By now, the method for optimizing automatic patient positioning in a medical imaging system and the medical imaging system according to the present invention have been described, and the processor and the computer-readable storage medium capable of implementing the method have also been introduced.

Via the present invention, the existing automatic patient positioning based on a 3D depth camera in a medical imaging system can be optimized, to improve centering accuracy, thereby improving imaging quality. By means of the present invention, the medical imaging system can reduce the possibility of re-performing a scout scan, and re-adjust the workbench height, thereby facilitating dose optimization, and achieving better workflow efficiency and productivity, better user experience and ease of use. With the improved centering effect, the medical imaging system is more friendly to pediatric hospitals, as automatic positioning of pediatric patients is not available or less effective in the prior art. The present invention is applicable to different on-site conditions, different camera mounting positions, and different user habits, and is flexibly applicable. Furthermore, the techniques of the present invention can be implemented as a fully automatic procedure, fast and intelligent, without the need for human or perceptual determination.

Some exemplary embodiments have been described above. However, it should be understood that various modifications can be made to the exemplary embodiments described above without departing from the spirit and scope of the present invention. For example, an appropriate result can be achieved if the described techniques are performed in a different order and/or if the components of the described system, architecture, device, or circuit are combined in a different manner and/or replaced or supplemented with additional components or equivalents thereof. Accordingly, the modified other embodiments also fall within the protection scope of the claims.

1. A method for automatic scan subject positioning in medical imaging, comprising the following steps:
acquiring scan information of a scan subject to be imaged and determining a scan subject scanning feature;
identifying, on the basis of the scan subject scanning feature, a corresponding offset characteristic value from a mapping table between scanning features and offset characteristic values; and
adjusting, on the basis of the corresponding offset characteristic value, a target position of the scan subject during the automatic scan subject positioning.

2. The method according to claim 1, wherein the mapping table is established via the following steps:
acquiring historical imaging scan data, the historical imaging scan data comprising historical scan information and historical offsets associated with a plurality of scanning procedures;
determining, from historical scan information of each scanning procedure, a scanning feature of a scan subject of the scanning procedure, the scanning feature of the scan subject comprising an anatomical structure category of the scan subject;
for the same scanning feature, calculating a statistical value of historical offsets thereof, and determining the statistical value or an inverse of the statistical value as an offset characteristic value corresponding to the scanning feature; and
generating a mapping table between different scanning features and offset characteristic values thereof.

3. The method according to claim 2, wherein the historical scan information comprises a scanning protocol and/or a scanning descriptor, and the anatomical structure category of the scan subject is determined on the basis of the scanning protocol and/or the scanning descriptor.

4. The method according to claim 2, wherein the scanning feature further comprises an age range, sex, and/or a body type of the scan subject, and the historical scan information comprises information for determining the age range, the sex, and/or the body type of the scan subject.

5. The method according to claim 2, wherein the historical scan information further comprises a positioning identifier for identifying whether manual positioning or automatic positioning is used in each scanning procedure, wherein the mapping table is generated only for scanning procedures using automatic positioning among the plurality of scanning procedures.

6. The method according to claim 1, wherein the scanning feature comprises an anatomical structure category, the scan information comprises a scanning protocol and/or a scanning descriptor, and the step of determining a scan subject scanning feature comprises: determining an anatomical structure category of the scan subject on the basis of the scanning protocol and/or the scanning descriptor.

7. The method according to claim 1, wherein the scanning feature comprises an anatomical structure category, the scan information comprises a scan range, and the step of determining a scan subject scanning feature comprises: identifying a plurality of landmarks of the scan subject by using a 3D camera, and determining an anatomical structure category of the scan subject on the basis of a positional relationship between at least one of the plurality of landmarks and the scan range.

8. The method according to claim 6, wherein the scanning feature further comprises an age range, sex, and/or a body type of the scan subject, and the scan information comprises information for determining the age range, the sex, and/or the body type of the scan subject.

9. The method according to claim 8, wherein the information for determining the body type of the scan subject comprises a BMI of the scan subject, or a body contour of the scan subject acquired by using a 3D camera.

10. The method according to claim 8, wherein age ranges of the scan subject comprise adult and pediatric, and body types of the scan subject comprises obese, normal, and thin.

11. The method according to claim 2, wherein an anatomical structure categories comprise head, chest, abdomen-pelvis, pelvis, lumbar spine, and heart.

12. The method according to claim 1, further comprising the following steps:
performing scanning and imaging on an anatomical structure of the scan subject according to the scan information by using a medical imaging system, so as to generate an image file, wherein the scan subject is moved in a horizontal direction during the scanning and imaging;
processing the image file to acquire a center point of the anatomical structure;
calculating a current offset in a vertical direction between the center point of the anatomical structure and an isocenter point of the medical imaging system; and
by using the scan subject scanning feature and the current offset, adjusting the offset characteristic value corresponding to the scan subject scanning feature in the mapping table.

13. The method according to claim 12, wherein the adjusting step comprises: performing statistical computation on the offset characteristic value corresponding to the scan subject scanning feature and the current offset, and determining a new offset characteristic value.

14. The method according to claim 1, wherein the scan subject to be imaged is movable in a horizontal direction and a vertical direction via a scan subject moving table, and the horizontal direction comprises a transverse direction and a longitudinal direction, wherein the step of adjusting a target position of the scan subject comprises adjusting a target position of the scan subject in the vertical direction or the transverse direction.

15. A medical imaging system, comprising:
a medical imaging apparatus, configured to perform scanning and imaging on a scan subject;
a scan subject moving table, configured to support the scan subject and movable in a horizontal direction and a vertical direction, the horizontal direction comprising a transverse direction and a longitudinal direction, wherein the scan subject moving table is moved in the longitudinal direction during the scanning and imaging;
a 3D depth camera, configured to acquire a body contour of the scan subject;
an automatic positioning module, configured to perform automatic positioning of the scan subject according to scan information of the scan subject and the acquired body contour; and
a positioning optimization module, configured to perform the following:
acquiring the scan information and determining a scan subject scanning feature;
identifying, on the basis of the scan subject scanning feature, a corresponding offset characteristic value from a mapping table between scanning features and offset characteristic values; and
adjusting, on the basis of the corresponding offset characteristic value, a target position of the scan subject in the automatic positioning.

16. The medical imaging system according to claim 15, wherein the mapping table is established via the following steps:
acquiring historical imaging scan data, the historical imaging scan data comprising historical scan information and historical offsets associated with a plurality of scanning procedures;
determining, from historical scan information of each scanning procedure, a scanning feature of a scan subject of the scanning procedure, the scanning feature of the scan subject comprising an anatomical structure category of the scan subject;
for the same scanning feature, calculating a statistical value of historical offsets thereof, and determining the statistical value or an inverse of the statistical value as an offset characteristic value corresponding to the scanning feature; and
generating a mapping table between different scanning features and offset characteristic values thereof.

17. The medical imaging system according to claim 16, wherein the historical scan information further comprises a positioning identifier for identifying whether manual positioning or automatic positioning is used in each scanning procedure, wherein the mapping table is generated only for scanning procedures using automatic positioning among the plurality of scanning procedures.

18. The medical imaging system according to claim 15, wherein the medical imaging apparatus is configured to perform scanning and imaging on an anatomical structure of the scan subject according to the scan information, so as to generate an image file, and the medical imaging system further comprises an adjustment module configured to perform the following:
processing the image file to acquire a center point of the anatomical structure;
calculating a current offset in a vertical direction between the center point of the anatomical structure and an isocenter point of the medical imaging system; and
by using the scan subject scanning feature and the current offset, adjusting the offset characteristic value corresponding to the scan subject scanning feature in the mapping table.

19. The medical imaging system according to claim 15, wherein the operation of adjusting a target position of the scan subject comprises adjusting a target position of the scan subject in the vertical direction or the transverse direction.
